(19) United States
(12) Patent Application Publication
(10) Pub. No.: US 2003/0212274 A1
(43) Pub. Date: Nov. 13, 2003

(54) Novel amorphous form of omeprazole salts

(76) Inventors: Bakthavathsalan Vijayaraghavan, Haryana (IN); Tarun Sharma, Bihar (IN); Naresh Kumar, Haryana (IN)

Correspondence Address: Jayadeep R Deshmukh, Ranbaxy Pharmaceuticals Inc, Suite 2100, 600 College Road East, Princeton, NJ, 08540, US

(21) Appl. No.: 10/276,875

(22) PCT Filed: May 11, 2001
(86) PCT No.: PCT/IB01/00820

(51) Int. Cl. C07F 7/28; C 07D 4 3/02

(52) U.S. Cl. 546/2; 546/273.7

(57) ABSTRACT

The present invention relates to novel amorphous form of salts and process for the preparation thereof.

### FIELD OF THE INVENTION

The present invention relates to a process for the preparation of novel amorphous form of salts of omeprazole including alkaline salts of the Formula II:

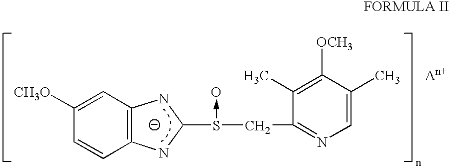

wherein n is 1, 2 or 4; Aⁿ⁺ is Li⁺, Na⁺, K⁺, Mg²⁺, Ca²⁺ etc, and process for the preparation thereof.

### BACKGROUND OF THE INVENTION

Chemically omeprazole is 5-methoxy-2-[(4-methoxy-3,5-dimethyl-2-pyridinyl)-methyl]sulfinyl-1H-benzimidazole. Omeprazole, covered in U.S. Pat. No. 4,255,431 is effective inhibitor of gastric acid secretion in mammals, by being inhibitor of H⁺, K⁺-ATP (proton pump) activity. The enzyme H⁺, K⁺-ATPase is responsible for gastric acid production and is located in the secretory membranes of the parietal cell. Omeprazole itself is not an active inhibitor of this enzyme, but it is transformed within the acid compartments of parietal cell into the active inhibitor, close to the enzyme. In a more general sense, omeprazole may be used for prevention and treatment of gastric acid related disorders and gastrointestinal inflammatory diseases in mammal and man, including e.g. gastritis, gastric ulcer and duodenal ulcer. Furthermore, omeprazole may be used for prevention and treatment of other gastrointestinal disorders where cytoprotective and/or gastric antisecretory effect is desirable e.g. in patients with gastrinomas, in patients with acute upper gastrointestinal bleeding, and in patients with history of chronic and excessive alcohol consumption. The term “omeprazole” as used in this specification designates the neutral form of the compound of the Formula I.

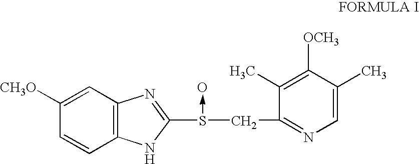

Active pharmaceutical ingredients that are easily destroyed in an acid medium (acid labile) such as omeprazole, create a special problem for formulations when it is required to provide a pharmaceutical form designated for oral administration. Omeprazole is susceptible to degradation/transformation in acid and neutral media. The half-life of degradation of omeprazole in water solutions at pH-values less than four is shorter than ten minutes. Also at neutral pH- values degradation proceeds rapidly, e.g. at pH=7 the half-life of omeprazole is about 14 hours, while at higher pH-values the stability in solution is much better [Ref. : Scand. J. Gastroenterology, 20 (suppl. 108), 113-120 (1985)]. Omeprazole also in the solid state is susceptible to degradation and is stabilized in mixtures with alkaline reacting compounds. The stability of omeprazole is also affected by moisture, heat, organic solvents and to some degree by light. Upon storage without any special precautions being taken, it is degraded at a rate which is higher than desired. At storage during accelerated conditions, that is at +37° C. and at a relative humidity of 80% for a period of 6 months, about 6% of the substance is converted to degradation products.

Certain salts of omeprazole including alkaline salts (K⁺, Li⁺, Na⁺, K⁺, Mg²⁺+, Ca²⁺ etc) and their manufacturing processes are described in U.S. Pat. No. 4,738,974. It has been found that alkaline salts of omeprazole of the structural Formula II, as shown in the accompanied drawings, wherein n is 1, 2 or 4; An⁺ is Li⁺, Na⁺, K⁺, Mg²⁺, Ca²⁺+ etc are more stable during storage than the corresponding neutral form of omeprazole. The salts of Formula II are also easier to handle than the neutral form in the manufacture of pharmaceutical dosage units. U.S. Pat. No. 5,900,424 claims an omeprazole magnesium salts having a degree of crystallinity higher than 70% and also describes a process of producing thereof.

However, the processes of isolation and purification through crystallization in full manufacturing scale of omeprazole salts described in U.S. Pat. Nos. 4,738,974 and 5,900,424 present one major problem in that the magnesium omeprazole salt crystals are very fragile, making pharmaceutical manufacturing processes utilizing this product less attractive in full scale production.

It is nevertheless desirable to obtain novel physical forms of omeprazole salts, which exhibit improved stability and make full-scale manufacturing feasible.

The term polymorphism include different physical forms, crystal forms, crystalline/liquid crystalline/non-crystalline (amorphous) forms. It has been observed that many antibiotics, antibacterials, tranquilizers etc, exhibit polymorphism and some/one of the polymorphic forms of a given drug exhibit superior bioavailability and consequently show much higher activity compared to other polymorphs. It has also been disclosed that the amorphous forms in a number of drugs exhibit different dissolution characteristics and in some cases different bioavailability patterns compared to the crystalline form Konne T., Chem. Pharm. Bull, 38, 2003 (1990). For some therapeutic indications one bioavailability pattern may be favoured over another. Cefuroxime axetil is the classical example of amorphous form exhibiting higher bioavailability.

### SUMMARY OF THE INVENTION

It is an object of the present invention to provide a new amorphous form of omeprazole salts. The process for the preparation said amorphous from uses conditions which are convenient to perform on a commercial scale and operationally safe.

A preferred group of omeprazole salts of Formula II are those wherein Aⁿ⁺ is Li⁺, Na⁺, K⁺, Mg²⁺,Ca²⁺, Ti⁴⁺. Further preferred salts are those wherein Aⁿ⁺ is Na⁺, Mg²⁺ and Ca²⁺. The Mg²⁺ salt is particularly more preferred.

Accordingly the present invention provides a process for the preparation of omeprazole salt particularly Mg²⁺ salt in new amorphous form which comprises reacting omeprazole with A (OR)ₙ in a non aqueous solvent such as an alcohol, ROH (only for alcoholates) or in an ether to get a solution followed by recovering amorphous form of omeprazole salt by spray drying, wherein R is an alkyl group containing 1-4 carbon atoms, n is 1, 2 or 4 and Aⁿ⁺ is Li⁺, Na⁺, K⁺, Mg²⁺, Ca²⁺, Ti⁴⁺ etc.

Illustrative examples of the radical R is CH₃, C₂H₅, n-C₃H₇, n-C₄H₉, i-C₄H₉, sec-C₄H₉ and tert.-C₄H₉. Preferably, the solvent may be selected from the group consisting of methanol, ethanol, isopropanol, n-butanol, tetrahydrofuran, 1,4-dioxane or mixture(s) thereof.

In accordance with the present invention, omeprazole salt is recovered from the solution in an amorphous form using spray-drying technique. The Mini-Spray Dryer (Model: Buchi 190, Switzerland) which has been used, operates on the principle of nozzle spraying product and the drying gas flows in the same direction. The drying gas can be air or inert gases such as nitrogen, argon, and carbon dioxide. Nitrogen gas is preferred in this case.

### BRIEF DESCRIPTION OF THE DRAWINGS

FIG. 1 represents the infrared spectra of the amorphous omeprazole magnesium of the present invention.

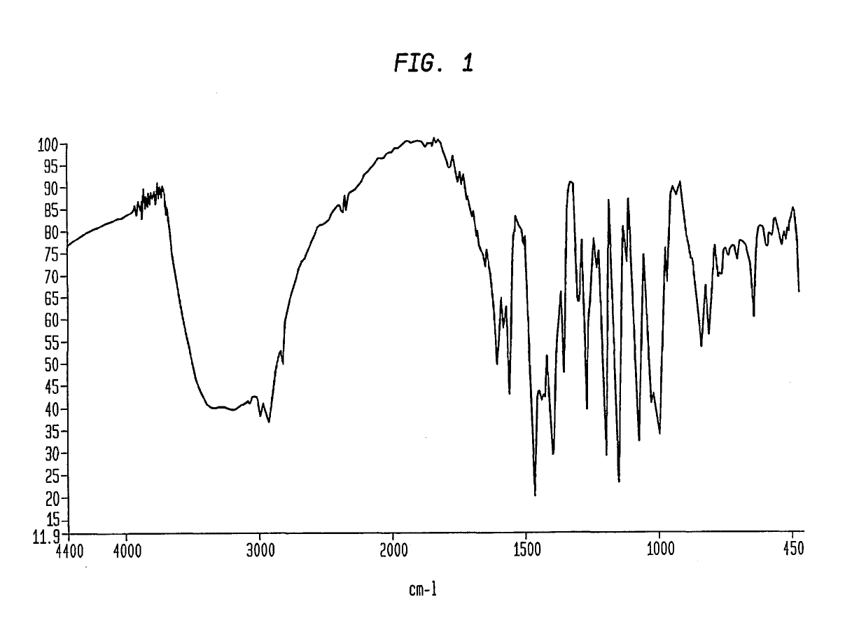

FIG. 2 represents the x-ray powder diffraction pattern of the amorphous omeprazole magnesium of the present invention.

FIG. 3 represents the infrared spectra of the crystalline prior art omeprazole magnesium.

FIG. 4 represent the x-ray powder diffraction pattern of crystalline prior art omeprazole magnesium.

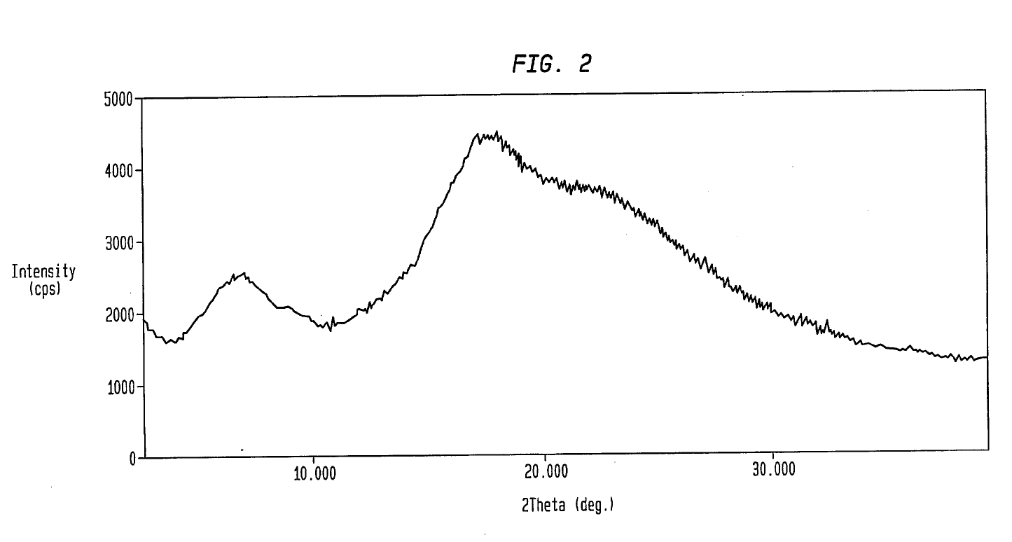

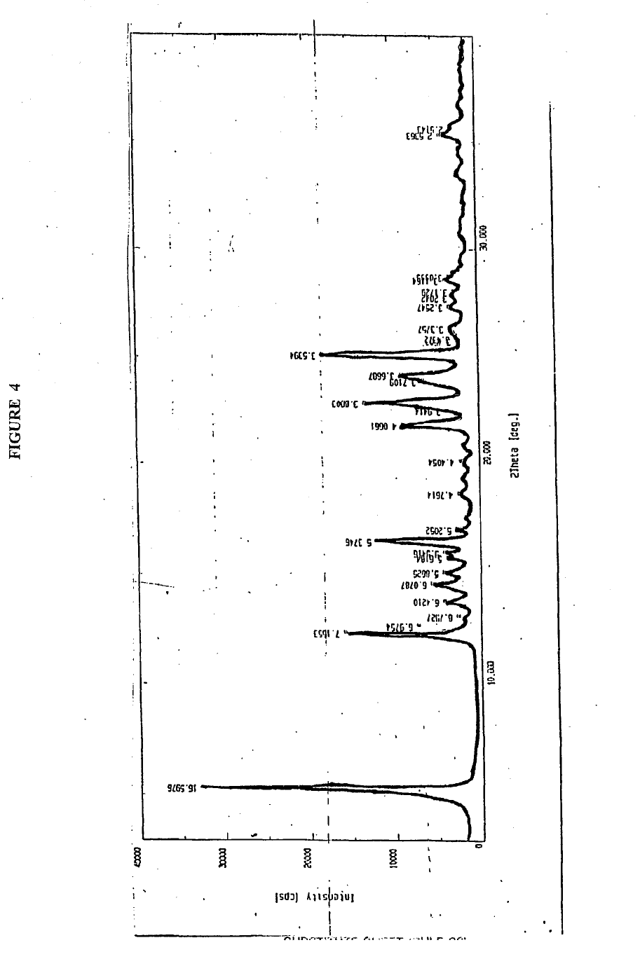

As seen in the Figures, x-ray powder diffraction patterns of the newly prepared form also gave a plain halo (FIG. 2) and show no peaks which are characteristic of the crystalline form of omeprazole magnesium, as shown in FIG. 4 of the accompanied drawings, thus demonstrating the amorphous nature of the product.

### DETAILED DESCRIPTION OF THE INVENTION

The present invention is illustrated by the following example, which is not intended to limit the effective scope of the claims.

### EXAMPLE 1

### Preparation of Amorphous Omeprazole Magnesium

Step A: Preparation of Omeprazole Base Slurry:

Omeprazole (100 gm) was added to methanol (900 ml) and stirred at 25-30° C. for about 30 minutes to get a uniform slurry.

Step B: Preparation of Fresh Magnesium Methoxide Solution

Methanol (500 ml) was heated to reflux at 65-67° C. and to it was added iodine crystals (100 mg). Magnesium turnings (3.86 gm) were added portionwise to the above solution during a period of 30 minutes maintaining reflux temperature of 65-67° C. The resulting reaction mixture was further refluxed for 30 minutes and was cooled to 30-35° C.

Step C: Preparation of Amorphous Omeprazole Magnesium

The freshly prepared magnesium methoxide solution (from Step A) was added into omeprazole slurry in methanol (from Step B) in one lot at 25-30° C. Stirred the resulting reaction mixture at 25-30° C. for about 1 hour. The clear solution thus obtained was subjected to spray drying in a mini-spray dryer (Buchi Model 190) at an inlet temperature of 60° C. with a feed rate of 15 ml per minute. Omeprazole magnesium (69 gm) in an amorphous form was thus isolated.

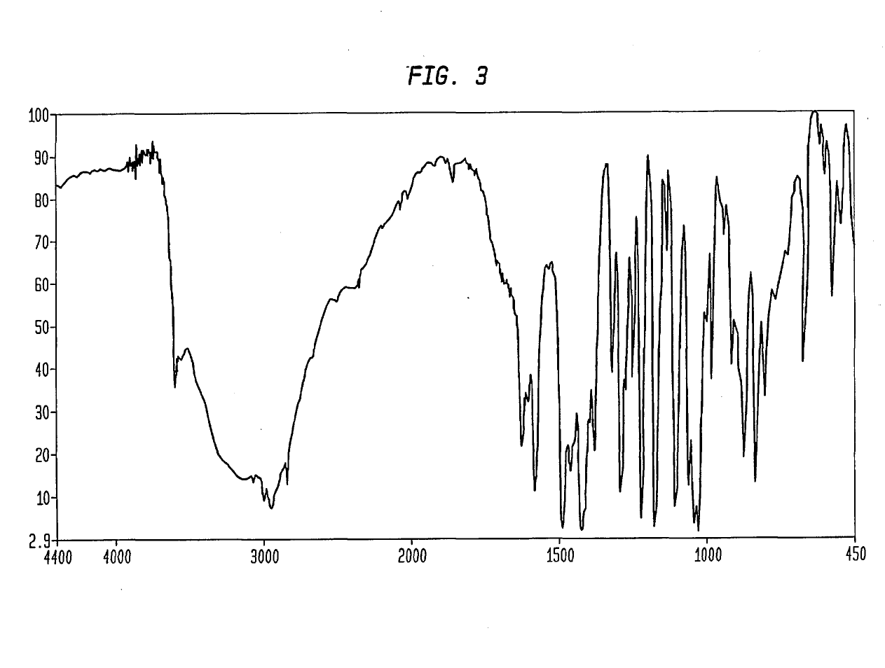

X-ray powder diffraction pattern as seen in FIG. 2, shows a plain halo thus demonstrating the amorphous nature of the product. Infra red spectrum in KBr, is different from the infra red spectrum for crystalline omeprazole magnesium, as shown in FIG. 3.

While the present invention has been described in terms of its specific embodiments, certain modifications and equivalents will be apparent to those skilled in the art and are intended to be included within the scope of the present invention.

We claim

1. Omeprazole salts of Formula II: 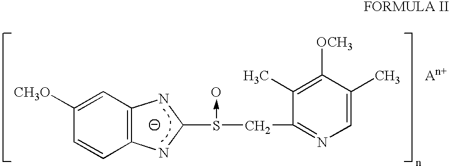
wherein n is 1, 2 or 4; Aⁿ⁺ is Li⁺, Na⁺, K⁺, Mg²⁺, Ca²⁺, or Ti⁴⁺, in an amorphous form.

2. The omeprazole salts of claim 1 wherein Aⁿ⁺ is Na⁺, K⁺, Mg²⁺, Ca²⁺.

3. The omeprazole salts of claim 2 wherein Aⁿ⁺ is Na⁺.

4. The omeprazole salts of claim 2 wherein Aⁿ⁺ is Mg²⁺.

5. The omeprazole salts of claim 2 wherein Aⁿ⁺ is Ca²⁺.

6. The omeprazole salts of claim 1 wherein R is CH₃, C₂H₅, n-C₃H₇, n-C₄H₉, i-C₄H₉, sec-C₄H₉ or tert.-C₄H₉.

7. The omeprazole salts of claim 6 wherein R is CH₃.

8. The omeprazole salts of claim 6 wherein R is C₂H₅.

9. The omeprazole salts of claim 1 wherein A(OR)ₙ is Mg(OCH₃)₂.

10. The omeprazole salts of claim 1 wherein A(OR)ₙ is Mg(OC₂ H₅)₂.

11. A process for the preparation of omeprazole salts of Formula II: 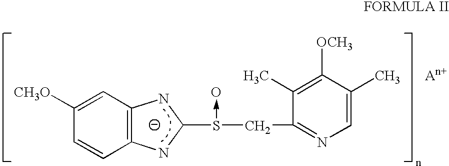
wherein n is 1, 2 or 4; Aⁿ⁺ is Li⁺, Na⁺, K⁺, Mg²⁺, Ca²⁺, or Ti⁴⁺, in an amorphous form which comprises reacting omeprazole with A(OR)ₙ in a non aqueous solvent wherein Aⁿ⁺ and n are the same as defined above, R is an alkyl group containing 1-4 carbon atoms, to get a solution and recovering amorphous omeprazole salt by spray drying.

12. The process of claim 11 wherein suitable non-aqueous solvent includes alcohols, ethers and mixture(s) thereof.

13. The process of claims 11 wherein the non-aqueous solvent is selected from methanol, ethanol, isopropanol, n-butanol, tetrahydrofuran, 1,4-dioxane or mixture(s) thereof.
